(19) United States
(12) Patent Application Publication
(10) Pub. No.: US 2008/0274990 A1
(43) Pub. Date: Nov. 6, 2008

(54) Cog47 Protein and S100beta Gene Polymorphism

(75) Inventors: Philippe Amouyel, Marcq en Baroeul (FR); Jean-Charles Lambert, Tourcoing (FR); Stephanie Ferreira, Lille (FR)

Correspondence Address: FOLEY AND LARDNER LLP;SUITE 500, 3000 K STREET NW, WASHINGTON, DC, 20007, US

(73) Assignee: GENOSCREEN

(21) Appl. No.: 11/629,495

(22) PCT Filed: Jun. 14, 2005
(86) PCT No.: PCT/FR2005/001480

§ 371 Date: May 23, 2007

(30) Foreign Application Priority Data
Jun. 14, 2004 (FR) 0406428

(51) Int. Cl. A61K 31/7105; C07K 14/435; C07K 16/00; G01N 33/53; A61K 31/711; A01K 67/027; C12N 15/00; C07H 21/04; C07H 21/02; C12Q 1/68

(52) U.S. Cl. 514/44; 530/350; 530/387.9; 435/7.1; 536/23.5; 435/6; 435/320.1; 800/18

(57) ABSTRACT

The invention concerns a Cog⁴⁷ protein and its polynucleotide, in particular associated with a predisposition to cognitive decline, to a conversion to dementia or to an already existing dementia, and the human S100β gene exhibiting a polymorphism associated with a predisposition to cognitive decline, to a conversion to dementia or to an already existing dementia. The invention also concerns a diagnostic method and a diagnostic kit.

The present invention concerns a Cog⁴⁷ protein and its polynucleotide, associated in particular with a predisposition to cognitive decline, to conversion to dementia or to already existing dementia, and the S100β gene exhibiting a polymorphism.

The invention also relates to a diagnostic method and to a diagnostic kit.

The dementias are neurodegenerative diseases of the central nervous system which correspond to a progressive attenuation of memory and other cognitive functions. After a variable-length period of deterioration of cognitive functions and personality, the disease leads to a complete state of dependence.

However, the same deteriorations are frequently observed to a lesser degree during so-called “normal” cerebral aging. Thus, for certain authors, dementia appears as an inevitable process beyond a certain age with a continuum between the decline of cognitive functions and dementia during the aging process. Thus, pathology would only be an exaggeration or an acceleration of this aging. This hypothesis of a continuum is one of the key elements that justifies a global or parallel approach to the study of the determinants of normal cerebral aging and of age-related cerebral pathologies. One of the principal arguments in favor of this hypothesis is the similarity between neuropathological lesions (senile plaques and neurofibrillary tangles) frequently observed in elderly people who do not have cognitive deterioration and lesions that are characteristic of Alzheimer's disease, an ailment that represents nearly 70% of dementias. In Alzheimer's disease, however, the density of these lesions is always much higher and their distribution broader.

If there is a continuum between the deterioration of cognitive functions and dementia, it would be logical for genetic-risk or environmental factors to be common to these two entities. The implication of the apolipoprotein E (APOE) gene as a genetic determinant of both Alzheimer's disease and of the global level of cognitive function in elderly subjects, as well as the presence of senile plaques in Alzheimer's patients and in the elderly, points in this direction. This suggests, therefore, that knowledge about certain genetic factors can make possible a better understanding of both the determinants of cognitive decline in the elderly and the appearance of dementia. However, the study of such genetic markers must be based on a strong physiopathological hypothesis.

It is known that astrocyte activation contributes to the maintenance and protection of the central nervous system, in particular following events that are acute and limited in time. However, within the framework of chronic and/or repeated lesions that induce systematic activation of these astrocytes, this adaptive repair mechanism can lead to a greater and faster progression of a lesion or a pathology. This chronic activation appears during so-called “normal” cerebral aging and takes on a considerable importance in pathologies such as Alzheimer's disease. These simple observations suggest, therefore, that the factors controlling the development of chronic astrocyte activation may also play a role between the deterioration of cognitive functions observed during aging and dementia.

Among the potential factors controlling this astrocyte response, protein S100β seems to be a special target for many reasons. At the physiological level, S100β, primarily produced by astrocytes, appears to be involved in brain development and maintenance but also in cognitive processes and their organization. Nevertheless, its importance with respect to these processes is still poorly defined and may even appear contradictory. Indeed, the injection of antibodies directed against S100β into the brains of rats leads to learning and memory disorders, whereas transgenic mice overexpressing S100β exhibit behavioral disorders similar to the disorders observed in certain dementias. This contradictory aspect may be explained by the fact that S100β is neuroprotective at nanomolar concentrations and neurotoxic at micromolar concentrations. At the pathological level, this latter observation may be essential. Indeed, astrocytes activated during the course of Alzheimer's disease markedly overexpress S100β. This protein is capable, by autocrine action, of promoting astrocyte proliferation and hypertrophy. Interestingly, however, the majority of the activated astrocytes overexpressing S100β are strongly associated with the presence of amyloid plaques, one of the two pathological markers for Alzheimer's disease.

Less specifically, this overexpression could also be a marker for the appearance of cognitive disorders or cerebral damage. Thus, overexpression of this protein is a risk indicator for cerebral damage in a fetus or a newborn. Similarly, an elevated concentration of S100β is a prognostic indicator of greater difficulty in recovering following cranial trauma or of a higher risk of the appearance of mild to severe cognitive disorders following a surgical procedure.

These data indicate, therefore, that the S100β protein is important in the establishment of cognitive functions and in their potential deterioration. In fact, any changes likely to modulate the biological functions of this protein may be essential to defining the risks of cognitive decline or dementia in the elderly as well as, for example, following a surgical procedure.

At present, the existing tools used to characterize cognitive decline, conversion to dementia or conversion to already existing dementia are based on very specialized psychometric tests whose use is reserved for practitioners. However, 10% to 37% of such diagnoses prove to be inaccurate. Moreover, such tests are relatively ineffective in the first stages of pathology due of the absence of objective and measurable clinical signs.

Thus, a certain number of biological tests, both genetic and proteomic, have been developed to overcome these deficiencies.

It was proposed that the ε4 allele of the APOE gene may be useful within this framework. However, it has since been shown that this genetic marker has a low predictive value for the level of cognitive decline or conversion to dementia.

At the protein level, tau proteins and amyloid peptides were quantified from samples of serum or cerebrospinal fluid. However, the results obtained showed that these biological measurements were no more effective than the psychometric tests, even if they made it possible to direct the diagnosis.

Alzheimer's disease, and more generally the dementias, constitute a major public health problem whose importance increases as human populations age.

In the current state of the art, there is no curative treatment for this disease. Its progression is leading toward massive desocialization related to temporospatial confusion and memory disorders that are incompatible with the autonomy necessary for an autonomous social life. The cost of such an affliction with respect to care and to dependence will be considerable and may, in the long term, overload the health care system.

Today, however, there are several symptomatic treatments based on improvement in cognitive and memory functions which act on certain neurotransmitters. These drugs belong to the class of acetylcholinesterase inhibitors which prolong the action of acetylcholine, an essential neurotransmitter. Four molecules are available today: Tacrine® (OTL Pharma), Donepezil® (Eisai), Rivastigmine® (Novartis) and Galantamine® (Janssen-Cilag). In addition, it has recently been proposed that the statins, molecules initially developed for the treatment of hypercholesterolemia, can also be used. Lastly, a novel molecule, memantine (Lundbeck), an anti-glutamatergic NMDA-receptor antagonist, is also protected.

These treatments, when patients are able to benefit from them, make it possible to delay desocialization to a certain extent. However, various work has shown that even in the absence of a curative treatment, the simple fact of delaying the appearance of the clinical signs of Alzheimer's disease by 5 years makes it possible to decrease the number of prevalent cases of this pathology by half, primarily due to the high concurrent mortality at these advanced ages.

Symptomatic treatments for Alzheimer's disease are only indicated in mild and moderate forms of dementia. Indeed, a certain neuronal potential must still be present so that these drugs can act. Also, so that patients can benefit best from these treatments, early identification of patients exhibiting discrete signs of dementia is essential.

Since the mid-1990s, the development of cholinesterase inhibitors has cleared the way for the therapeutic care of patients exhibiting mild to moderate forms of Alzheimer's disease. Currently, several therapeutic trials are under way regarding so-called “Mild Cognitive Impairment.” Diagnosing this moderate effect on the intellect, based on tests of cognitive functions, is still imprecise and the risk of mistakenly treating patients who are not suffering from dementia and who have only minor disorders, related to a depressive episode for example, is high.

Much work has shown that the quantification of plasma S100β is a very good indicator of the extent of post-traumatic cerebral lesions in both children and adults. This quantification is a very good marker for recovery level and period as well as for risks of complications related to this type of pathology. For example, monitoring the concentration of S100β is proposed for predicting the development of secondary intracranial hypertension following cranial trauma, as this complication is potentially fatal.

Similarly, S100β concentration is more specifically associated with the level of deterioration of the hematoencephalic barrier, the presence of cerebral tumors and metastases. In addition, S100β level correlates with tumor size and thus could be used as an early marker for cerebral tumors.

Lastly, S100β level is a very early marker for cerebral lesions in a fetus or newborn, whereas standard evaluation methods do not make detection of these types of lesions possible. It has thus been proposed that S100β can be used as a marker for cerebral lesions in perinatal medicine.

S100β quantity is also a sensitive marker for risks of complications following cardiac surgery. Thus, this quantification is predictive of the risk of the occurrence of a stroke, this pathology being a recurrent problem following open-heart surgery. It is also correlated with the extent of the stroke and thus with the risk of death for the patient having suffered such a complication. In addition, S100β concentration is an indicator of cognitive decline following this type of operation and of the recovery level of these cognitive functions.

Finally, measurement of S100β level is an excellent marker for the success of clot lysis following a stroke.

It has already been disclosed that S100β can be used as a marker for cerebral tumors. However, the development of other cancerous lesions can be monitored by the measurement of circulating Sloop. Thus, this appears to be the most suitable measurement for following the appearance and development of metastases arising from melanomas.

U.S. Pat. No. 6,555,327 divulges a quantitative determination method for S100β and S100ββ protein for diagnosing brain traumas, neurological complications following bypass surgery and malignant melanomas.

International patent application WO 00/26668 divulges a method for diagnosing and predicting cancer in a subject by detecting at least one S100 protein among S100AG, S100A7, S100A8 and S100A9 using an immunological test.

International patent application WO 98/01471 divulges a peptide containing at least one fragment of the S100β protein. It also divulges a method for determining the presence of said peptide in a sample to diagnose neurological diseases generally.

International patent application WO 02/089656 divulges a method for diagnosing a clinical event in patient tissues, more particularly myocardial infarction, by the detection of the S100β protein.

U.S. Pat. No. 5,849,528 divulges two human S100 proteins (S100T1 and S100T2). The document also divulges a polynucleotide coding for said protein, as well as a composition containing said polynucleotide for diagnosing the progression of neuropathological changes in Alzheimer's disease.

Lastly, US patent application 2002/160,425 divulges chiefly a method for diagnosing and differentiating dementia related to Alzheimer's in a mammal by using an antibody specifically linked to a biochemical marker for Alzheimer's disease, such as S100β protein, and by determining the presence of said marker.

The diagnostic methods described above, which are specific for Alzheimer's disease, do not make it possible to diagnose the disease sufficiently early so that a suitable treatment can be genuinely effective in the long term.

A simple, routine diagnostic tool capable of being performed on circulating blood that makes it possible to improve diagnostic certainty and to guarantee therapeutic indications before the appearance, for example, of advanced symptoms of Alzheimer's disease is thus clearly needed.

Thus the goal of the inventors was to develop a simple, routine diagnostic tool capable of being performed on circulating blood that makes it possible to diagnose a biological marker of cognitive decline, of conversion to dementia or of conversion to already existing dementia.

Thus an object of the present invention is a protein named Cog⁴⁷ represented by the sequence SEQ ID NO 1 having a sequence of amino acids that is different than that of the normal S100β protein.

The examples which follow demonstrate that the Cog⁴⁷ protein is notably associated with a predisposition to cognitive decline, to conversion to dementia or to already existing dementia. These examples should not exclude the implication of Cog⁴⁷ protein in other pathologies.

In the present invention, the expression “cognitive decline” means the non-pathological progressive loss of mnestic and higher cognitive functions.

In the present invention, the expression “conversion to dementia” means the appearance of an event corresponding to the clinical appearance of dementia, from an asymptomatic or subclinical state to a symptomatic or pathological state.

In the present invention, the expression “already existing dementia” means a prevalent case of dementia upon inclusion in the study or at the time of diagnosis.

The present invention also has as an object a monoclonal or polyclonal antibody, or a fragment of said antibody, bound specifically on the Cog⁴⁷ protein by affinity with the sequence of amino acids that is different than that of the normal S100β protein, and the use of said polyclonal or monoclonal antibody, or fragment of said antibody, for the implementation of an in vitro immunological test in humans.

In the present invention, the expression “affinity with the sequence” means recognition by the antibody of the sequence of amino acids, either by recognition of the alignment of said amino acids or by recognition of the spatial conformation of said amino acids.

In the present invention, the expression “fragment of said antibody” means the part of said antibody that allows at least the recognition of the epitope of the Cog⁴⁷ protein, said epitope being present at the level of the sequence of amino acids that is different than that of the normal S100β protein. At a minimum the fragment contains the corresponding paratope(s).

The present invention also has as an object the use of a protein named Cog⁴⁷ such as previously defined for the in vitro production of antibodies that specifically bind to said protein.

The present invention in addition has as an object a method for determining the presence of a Cog⁴⁷ protein represented by the sequence SEQ ID NO 1 in a human sample comprising the following steps:
allowing the sample to be analyzed to react with a first antibody such as previously described,
allowing the sample to be analyzed to react with a second antibody specifically directed against the antibody such as previously described,
washing, and
detecting the quantity of Cog⁴⁷ protein in the sample.

Preferably, in the method previously described the antibody is a monoclonal antibody.

By “human sample” is meant any body fluid or tissue taken from a human, such as, for example, a sample of blood or cerebrospinal fluid.

Another object of the invention relates to the isolated and purified mRNA polynucleotide encoding the Cog⁴⁷ protein, represented by the sequence SEQ ID NO 2.

Still another object of the invention relates to an interfering mRNA polynucleotide that specifically targets the mRNA previously described, as well as a DNA polynucleotide capable of expressing said interfering mRNA polynucleotide. The invention also relates to said interfering mRNA polynucleotide or DNA polynucleotide according to the invention for therapeutic use for inhibiting or decreasing expression of the Cog⁴⁷ protein.

In the present invention, “therapeutic use” means “use as a drug.”

The therapeutic indications are principally cognitive decline, conversion to dementia and conversion to already existing dementia. Other therapeutic indications that are currently unknown are also comprised in the use as a drug.

RNA interference (RNAi) technology makes it possible to silence a gene in a cell and offers many therapeutic applications.

Interfering RNA can be obtained either by the administration of double-stranded RNA which is degraded in the cell into small interfering RNA (siRNA), or by the administration of double-stranded DNA in the DNA-directed RNA interference (ddRNAi) technique which triggers the production of siRNA. This technology is adequately described in the scientific literature and is the object of patent application filings (SIRNA THERAPEUTICS, BENITEC, etc.).

The present invention in addition has as an object a Cog⁴⁷ protein expression vector comprising at least the DNA sequence complementary to the sequence SEQ ID NO 2 and elements allowing the expression of said protein in a prokaryotic host cell with the purpose of producing a recombinant protein.

The present invention also has as an object a diagnostic kit for determining the presence of the Cog⁴⁷ protein represented by the sequence SEQ ID NO 1 in a human sample, comprising a Cog⁴⁷ protein such as previously described and/or polyclonal or monoclonal antibodies, or fragments thereof, such as previously described, and/or an oligonucleotide comprising 20 to 25 bp complementary to the mRNA such as previously described.

Another object of the invention relates to the use of the polynucleotide mRNA such as previously described, or a fragment thereof, for the in vitro diagnosis of a predisposition to cognitive decline, a conversion to dementia and an already existing dementia.

Another object of the invention relates to a transgenic mouse comprising in its genome the nucleotide sequence required to express the COG⁴⁷ protein according to the sequence SEQ ID NO 1 with the purpose of developing a model for studying the involvement of Cog⁴⁷ in pathological processes and potential therapeutic targets.

The present invention also has as an object an isolated human S100β gene represented by the sequence SEQ ID NO 3, wherein said gene exhibits at least one polymorphism associated with a predisposition to cognitive decline, a conversion to dementia or an already existing dementia.

The examples which follow demonstrate that certain polymorphisms of the human S100β gene are associated with a predisposition to cognitive decline, to conversion to dementia or to already existing dementia.

In a highly preferential embodiment of the invention, the polymorphism of the S100β gene is characterized in that the polymorphism is located at the thymidine nucleotide that is −100 with respect to the transcription initiation site of said S100β gene. Preferably still, the gene polymorphism is characterized in that the thymidine nucleotide is replaced by a cytosine nucleotide.

The present invention also has as an object the use of the human S100β gene such as previously defined, or a fragment thereof, for diagnosing a predisposition to cognitive decline, a conversion to dementia or an already existing dementia.

The present invention in addition has as an object the use of the human S100β gene represented by the sequence SEQ ID NO 3, or a fragment thereof, for diagnosing a predisposition to cognitive decline, a conversion to dementia or to already existing dementia, wherein the polymorphism is located at the guanidine nucleotide that is +2766 with respect to the transcription initiation site of said gene.

In the present invention, the expression “a fragment thereof” when associated with “gene” means a part of said gene.

In a preferential embodiment of the invention, in the use of the S100β gene previously described, the guanidine nucleotide is replaced by a cytosine nucleotide.

The present invention also has as an object the use of the human S100β gene represented by the sequence SEQ ID NO 3, or a fragment thereof, for diagnosing a predisposition to cognitive decline, a conversion to dementia or an already existing dementia, wherein the polymorphism is located at the adenine nucleotide that is +2963 with respect to the transcription initiation site of said gene.

In a preferential embodiment of the invention, in the use of the S100β gene previously described, the guanidine nucleotide is replaced by an adenine nucleotide.

The present invention also has as an object the use of the human S100β gene represented by the sequence SEQ ID NO 3, or a fragment thereof, for diagnosing a predisposition to cognitive decline, a conversion to dementia or an already existing dementia, wherein the polymorphism is located at the guanidine nucleotide that is +3942 with respect to the transcription initiation site of said gene.

In a preferential embodiment of the invention, in the use of the S100β gene previously described, the guanidine nucleotide is replaced by an adenine nucleotide.

Another object of the present invention relates to a diagnostic kit for determining the presence in a sample of a polymorphism of the S100β gene represented by the sequence SEQ ID NO 3 associated with a predisposition to cognitive decline, to conversion to dementia or to already existing dementia, comprising one or more oligonucleotides of 20 to 25 bp complementary to the polymorphisms such as previously described.

The present invention also has for an object an in vitro diagnostic method of the predisposition to cognitive decline, to conversion to dementia or to already existing dementia in a human comprising the determination of the presence of a polymorphism such as previously described and/or of proteins associated with said polymorphism, notably the Cog⁴⁷ protein.

According to a final aspect, the present invention has as an object an in vitro method for identifying a molecule capable of interacting with the Cog⁴⁷ protein comprising the following steps:
a) bringing together a suitable cellular model with the Cog⁴⁷ protein and said molecule to be identified in order to characterize the quantity of phosphorylated tau proteins using at least one marker specific for tau protein phosphorylation, and/or
b) bringing together a suitable cellular model with the Cog⁴⁷ protein and said molecule to be identified in order to characterize the level of expression of at least one marker for microglial inflammation,
c) when:
   the quantity of phosphorylated tau proteins characterized in step a) is less than the quantity of phosphorylated tau proteins characterized in the absence of said molecule, and/or
   the level of expression of said at least one marker for microglial inflammation characterized in step b) is less than the level of expression of said marker characterized in the absence of said molecule,
identifying said molecule capable of interacting with the Cog⁴⁷ protein.

The molecule to be identified that is capable of modulating the activity of the Cog⁴⁷ protein can be a chemical or biological molecule (isolated, recombinant or synthetic).

The cellular model suitable for characterizing the quantity of phosphorylated tau proteins is well known to those skilled in the art and is, in general, a neuronal cellular model.

The cellular model suitable for characterizing the level of expression of said at least one marker for microglial inflammation is, in general, a model comprised of microglial cells.

However, those skilled in the art may envisage modifying these two cellular models, perhaps even combining them, in order to characterize the quantity of phosphorylated tau proteins and the level of expression of the marker for microglial inflammation simultaneously.

Each of these cellular models is brought together with the Cog⁴⁷ protein and the molecule to be identified.

When the neuronal cellular model is used to characterize the quantity of phosphorylated tau proteins, the bringing together of the Cog⁴⁷ protein consists, in general, of adding a solution of this Cog⁴⁷ protein to said cellular model.

When the cellular model used to characterize the level of expression of the marker for microglial inflammation is a model comprised of microglial cells, it is generally preferred to transform these microglial cells in a preliminary step using a nucleic acid coding for the Cog⁴⁷ protein.

According to a preferred embodiment, said at least one marker specific for tau protein phosphorylation is an antibody directed against a phosphorylated epitope of the tau protein, this epitope being characteristic of neuronal degeneration. Preferably, at least two, three or four markers specific for phosphorylation are used.

Other specific markers for said phosphorylation that are well known to those skilled in the art can be used. Without being limiting in any way, the monoclonal antibodies AD2 and AT8 can be cited as examples of antibodies directed against a phosphorylated epitope of the tau protein.

According to another preferred embodiment, said at least one marker for microglial inflammation is a cytokine.

Preferably, the level of expression of at least two, preferably at least three, four, or at least five cytokines is characterized.

Without being limiting in any way, tumor necrosis factor α (TNFα) and interleukin (IL6) can be cited as examples of cytokines.

Optionally, the morphology of the microglial cells is characterized in addition to the characterization of the level of expression of said at least one marker for inflammation.

According to another alternative, microglial inflammation can be characterized at the tissue level using a ferritin staining.

In addition to the preceding provisions, the present invention comprises still other provisions which will arise in the further description which follows, which refers to the examples as well as to the appended drawings in which:

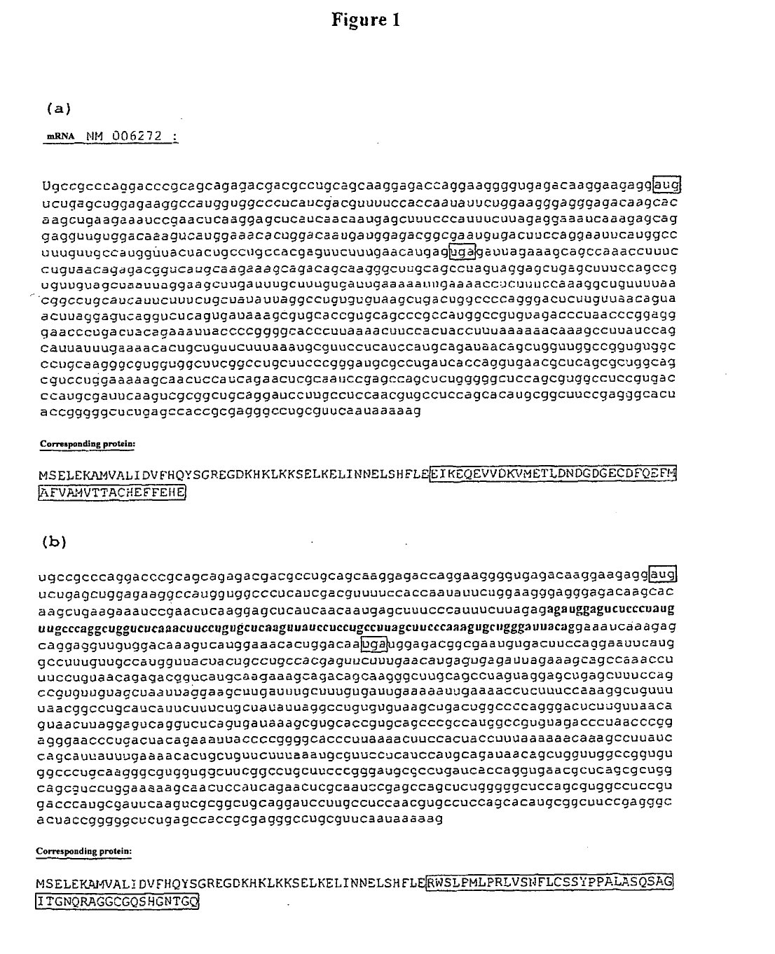

FIG. 1(a) presents the sequence of the mRNA and of the protein corresponding to the normal S100β isoform, 1(b) presents the sequence of the mRNA and of the protein corresponding to the sequence of the Cog⁴⁷ isoform from total mRNA;

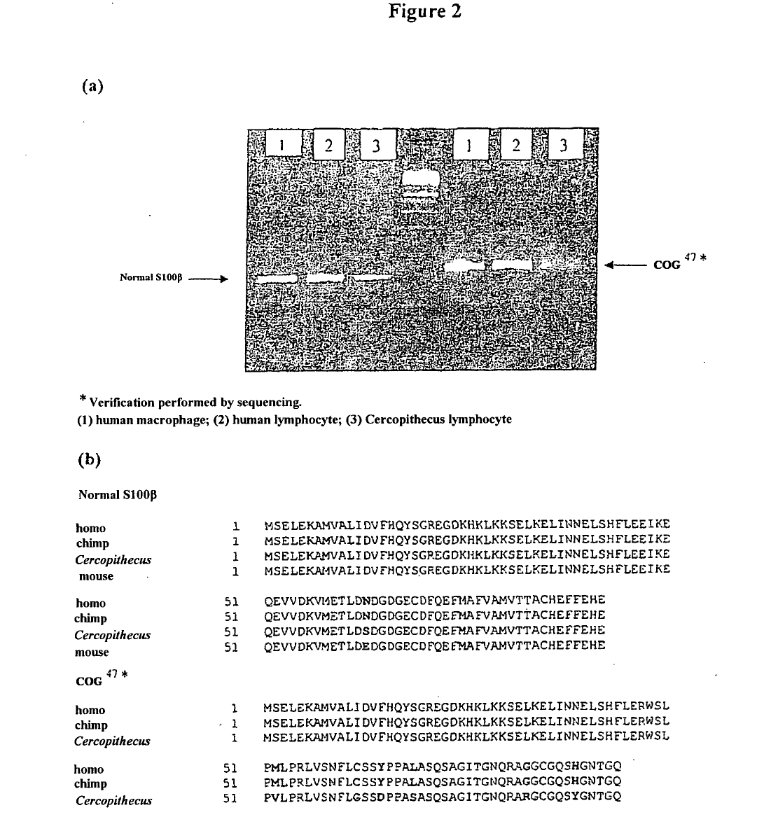

FIG. 2(a) presents the amplification by RT-PCR of the mRNA corresponding to normal S100β and Cog⁴⁷ from human macrophages and lymphocytes and from Cercopithecus lymphocytes; FIG. 2(b) presents the inter-species comparison of the protein sequences of normal S100β and Cog⁴⁷ (after genomic DNA sequencing). The amino acids that differ between the species studied are indicated in gray;

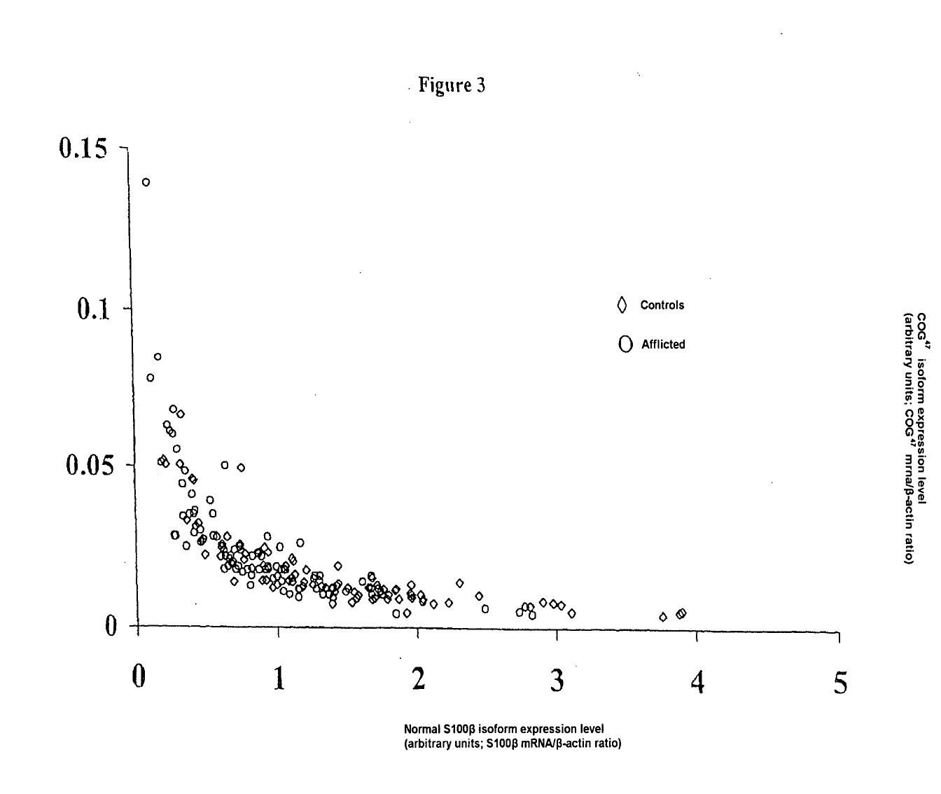

FIG. 3 presents the levels of expression of the isoforms of S100β and Cog⁴⁷ in the cerebral tissue of 85 patients suffering from Alzheimer's disease and 90 controls;

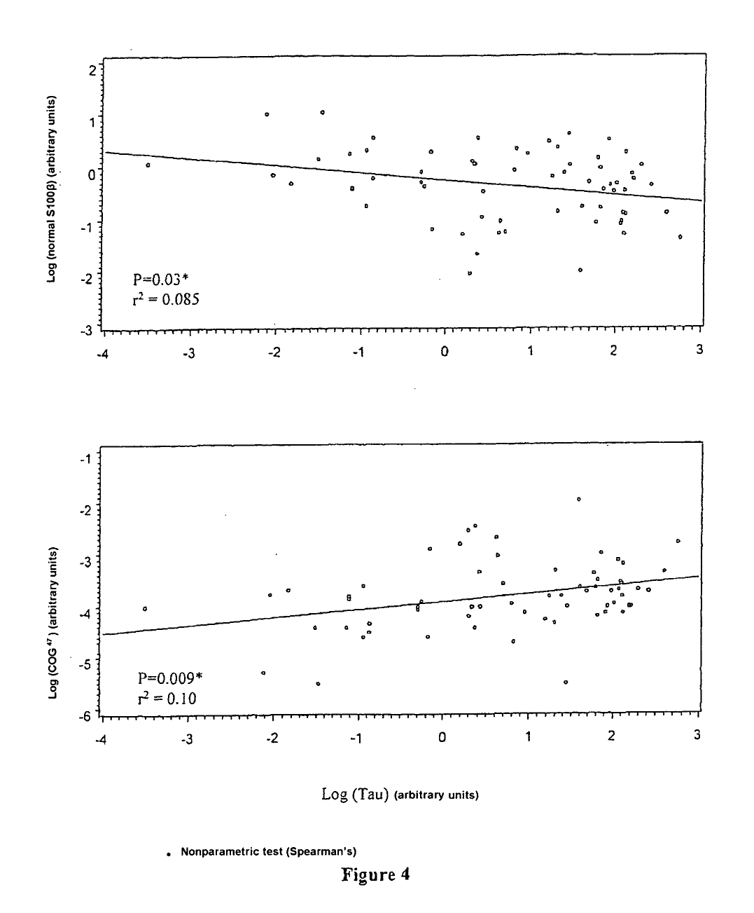

FIG. 4 presents the correlation between the level of expression of S100β and Cog⁴⁷ and the level of neurofibrillary degeneration;

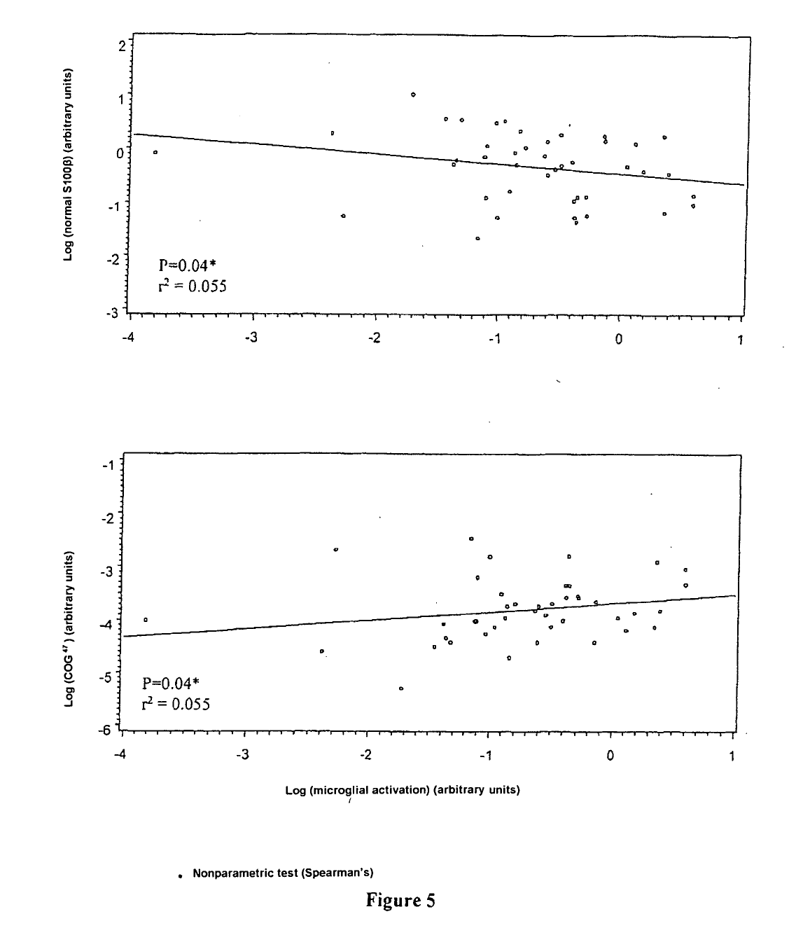

FIG. 5 presents the correlation between the level of expression of S100β and Cog⁴⁷ and microglial inflammation.

The examples which follow makes it possible to illustrate the optimal embodiment of the invention, but in no way should they be interpreted as limiting the scope of the claims.

### EXAMPLE

### Study of the Polymorphism of the S100β Gene and of the Cog⁴⁷ Protein Associated with a Predisposition to Cognitive Decline, to Conversion to Dementia or to Conversion to Already Existing Dementia

1. Materials and Methods:

ELDNOR Population

The ELDNOR (ELDerly in the NORth of France) study was carried out in the département du Nord, an administrative region of France, between March and September 1993. A random sample of 1055 individuals of both sexes between 60 and 105 years of age was recruited from retirement homes. One doctor performed all interviews and took all blood samples. At the beginning of the interview, all subjects received a verbal description of the study and signed an informed consent statement. For those who were mentally incompetent, their descendents or legal guardians were consulted. The Ethics Committee of the local university approved the study. An MMSE was performed for all subjects. 187 individuals were diagnosed with dementia.

Population of Limeil-Brévannes

125 cases of dementia (28.7% men, 85.5 years of age) were recruited from the long-term care gerontology department of the Limeil-Brévannes Hospital. (France). Dementia was identified according to DSM-III-R criteria. 36 individuals exhibiting cognitive decline without dementia criteria were also recruited. Brains (n=167, 50.9% men, 79.6 years of age) arising from standard autopsies performed at the Municipal Hospices of Strasbourg (eastern France) were used as controls for this study. Recruitment was designed in order to exclude cases of dementia and patients suffering from neurological pathologies. The majority of the subjects were admitted to the emergency services less than 48 hours before death and lived at home before their admission. All of the studies were approved by the local Ethics Committees.

Alzheimer's Population

The control and Alzheimer's samples were Caucasian, recruited in the north of France (Alzheimer's cases: n=589, age=72.3 years, age at onset=69.4 years, 39.5% men; controls: n=663, age=72.5±7.9 years, 36% men). The probable diagnosis of Alzheimer's disease was established according to DSM-III-R and NINCDS-ADRDA criteria. The Caucasian controls were defined as subjects without DSM-III-R dementia criteria and with complete cognitive functions. Each individual, or his or her relation or legal guardian, gave informed consent.

Brain Samples

85 brains of patients suffering from Alzheimer's disease were obtained from a bank of 114 patients with definitive forms of the pathology recruited in Manchester (United Kingdom) and its nearby suburbs during the years 1986-2001 (average age at death=73.1±9.1 years; average age at onset=65.9±10.3 years; 51% males). All of the diagnoses were made according to CERAD neuropathological criteria.

The quantity of amyloid plaques Aβₙ₋₄₀, Aβₙ₋₄₂₍₄₃₎ and total Aβ (Aβₙ₋₄₀+Aβₙ₋₄₂₍₄₃₎) was measured by image analysis of the frontal cortex (Brodmann areas 8/9) as previously described. Tau load was determined in 86 samples by a standard method using the monoclonal antibody AT8 (Innogenetics, Belgium) as the primary antibody. The assay for ferritin, as a marker for activated microglia, was carried out on 72 brains by using a standard method, incubation with a primary antibody against ferritin (Sigma, R-V) overnight at 4° C. (at a dilution of 1:750). Ferritin immunoreactivity was then visualized by the addition of 3,3-diaminobenzidene (DAB).

The 90 control brains (79.4±6.1 years of age; 43% males) come from a first group of 190 brains obtained from standard autopsies performed at the Municipal Hospices of Strasbourg (France). Recruitment was designed to exclude cases of dementia. The individuals were recruited from a general hospital and not from geriatric institutions in which a majority of patients may have dementia. The majority of the subjects were admitted to the emergency services less than 48 hours before death and lived at home before their admission. Cases whose autopsy revealed neurological pathologies were excluded. Neuropathology criteria were applied to define Braak stages and were in conformity with CERAD neuropathological criteria.

Sequencing and Genotyping

All exons and intron/exon junctions, as well as the proximal promoter and intron 3 of the S100β gene, were examined in 32 controls using D-HPLC (denaturing high-performance liquid chromatography) analysis to demonstrate sequence variations. D-HPLC, also known as temperature-mediated heteroduplex analysis, is an extremely sensitive technique for detecting DNA sequence variations, in particular single base substitutions. All of the variants identified by D-HPLC were confirmed by sequencing. PCR products were sequenced directly using the Taq dye Terminator sequencing kit (Perkin Elmer Biosystems, Foster City, Calif.).

The rs2000403 genotype was determined by amplification of a fragment using 5′-actctgaaccattcacggtg-31 and 5′-gtctctcaccaagccctatt-3′ primers. The genotype was then determined by enzymatic digestion (NIa III).

Real-Time RT-PCR

Total brain RNA was extracted from frozen tissue using a protocol based on the use of phenol-chloroform (TRIzol® reagent, Invitrogen). The quality of the total RNA was evaluated using an Agilent 2100 bioanalyzer and the 28S:18S ribosomal RNA ratio was estimated systematically (range between 0.0 and 1.8). Real-time reverse transcription and PCR amplification was performed with 30 ng of cDNA/RNA using Taqman technology, thus making it possible to co-amplify the cDNA representing the S100β, Cog⁴⁷ and β-actin genes as described by the manufacturer (Applied Biosystems).

Statistical Analyses

Statistical analyses were carried out using the SAS v. 7.0 statistical software (SAS Institute Inc., Cary, N.C.). The level of total RNA degradation (estimated by the 28S:18S rRNA ratio) was not identified as a confounding factor for the S100β or Cog⁴⁷ expression levels measured (using a linear regression model). This degradation level was, however, included in the multivariate analysis of covariance using a general linear model for the comparison of the quantity of mRNA between the cases of Alzheimer's and the controls.

Tau load, microglial activation level and mRNA quantifications were log-transformed to obtain a normal distribution. The effect of the rs2000403 polymorphism on mRNA level was examined using the nonparametric Wilcoxon test. The odds ratios, or ORs (which allow the evaluation of a risk for Alzheimer's disease for a high or low level of expression of Cog⁴⁷ or Sloop) were evaluated by logistic regression, the subpopulations having been defined using the median mRNA level.

Univariate analyses were performed using Pearson's x² test. In the multivariate analyses, the genotype of the rs2000403 polymorphism was coded by dichotomizing the genotypes according to the presence or absence of an A allele (GG against AA+GA). The effect of these variables on the disease was evaluated by a logistic linear regression model adjusted for age and sex. Pair-wise linkage-disequilibrium coefficients were estimated for the control samples. Extended haplotype frequencies of the two markers were estimated using the myriad haplotype frequency algorithm described by McLean et al.

2. Study of the Polymorphism of the S100β Gene and the Cog⁴⁷ Protein:

By comparing EST sequences available on the University of California Santa Cruz Web site (http://genome.ucsc.edu), the existence of an unknown exon in the S100β gene, until now thought to be comprised of 3 exons, was demonstrated. A fourth exon was demonstrated by RT-PCR, thus making it possible to establish a new mRNA sequence (FIG. 1(b)).

The novel isoform, named Cog⁴⁷, differs from the previously-known S100β isoform by the last 48 amino acids. The sequence corresponding to this exon was not found in mouse genomic DNA. On the other hand, it was observed in both chimpanzee and Cercopithecus, which express this exon in lymphocytes (FIG. 2(a)). The protein sequence of the normal S100β isoform or the Cog⁴⁷ isoform are identical in man or chimpanzee. However, this protein sequence differs by one amino acid and six amino acids for S100β and Cog⁴⁷, respectively, in Cercopithecus. These associated data, due the fact that demonstration of this exon sequence in the lemur has not been successful, suggest that the Cog⁴⁷ protein is recent in evolutionary terms.

Expression levels of S100β and Cog⁴⁷ isoforms were measured in the cerebral tissue of 85 patients suffering from Alzheimer's disease and 90 controls. Using semi-quantitative RT-PCR, it was observed that the Cog⁴⁷ expression level appeared inversely correlated to the S100β expression level: the lower the level of S100β, the higher the level of Cog⁴⁷ (FIG. 3). This result suggests interdependent production mechanisms for these two isoforms. Reinforcing this possibility, it was also observed that the rate of S100β expression was 37% lower among the afflicted compared to the controls, whereas that of Cog⁴⁷ was almost 41% higher among these same afflicted patients (table 1).

TABLE 1
Expression level of various mRNA arising from the
S100β gene in the control population and the
population suffering from Alzheimer's disease.

| | Controls | Afflicted | | |
|---|---|---|---|---|
| n | 90 | 84 | | p |
| Normal S100β | 1.19 | 0.74 | −37.2% | 0.0001 |
| mRNA | (0.63-2.24) | (0.38-1.47) | | |
| Cog⁴⁷ mRNA | 0.015 | 0.021 | +40.9% | 0.0004 |
| | (0.008-0.026) | (0.011-0.040) | | |
| Cog⁴⁷/S100β | 0.012 | 0.028 | +124.3% | 0.0001 |
| ratio | (0.004-0.041) | (0.007-0.104) | | |

Individuals exhibiting a high level of Cog⁴⁷ expression have a three-fold higher risk of developing Alzheimer's disease than those with a lower expression level of this isoform (table 2).

TABLE 2
Estimation of the risk of developing Alzheimer's
disease as a function of the mRNA levels of normal
S100β and Cog⁴⁷ in the frontal lobe.

| | OR | 95% CI | p |
|---|---|---|---|
| Elevated level of normal S100β mRNA | 0.33 | 0.18-0.63 | 0.0006 |
| Elevated level of Cog⁴⁷ mRNA | 3.11 | 1.67-5.77 | 0.0003 |
| Elevated COG⁴⁷/S100β ratio | 3.44 | 1.84-6.41 | 0.0001 |

(Median value used to differentiate the high and low expression levels.)

Consistent with these results, a positive correlation between Cog⁴⁷ expression level and neurofibrillary degeneration level (FIG. 4) or microglial inflammation level (FIG. 5) was demonstrated in the cerebral tissue of the afflicted patients, this expression level explaining 10% and 5.5%, respectively, of the variance of these pathological markers.

All the preceding data, therefore, appear to indicate that, beyond the fact that S100β and Cog⁴⁷ expression levels are markers for Alzheimer's disease, Cog⁴⁷ is a determinant of the pathology.

It is apparent that genetic variations controlling Cog⁴⁷ and/or S100β expression levels are associated with Cognitive decline and with the risk of developing dementia such as Alzheimer's disease. This hypothesis was confirmed by a systematic search for polymorphisms by D-HPLC and sequencing, carried out on the promoter, on the exons as well as on intron 3 (including the novel exon characteristic of Cog⁴⁷) of the S100β gene.

Thus, the existence of a certain number of polymorphisms was confirmed and the existence of novel polymorphisms was discovered (table 3).

TABLE 3
Polymorphisms characterized by D-HPLC
and sequencing in the S100β gene

| Number | Position | Location | Changes^{a} | Ref. | Frequency |
|---|---|---|---|---|---|
| 1 | −1358 | promoter | C/T | rs3788266 | 47.1% |
| 2 | −1132 | promoter | T/C | unknown | <1% |
| 3 | −949 | promoter | G/C | rs283965 | 43.0% |
| 4 | −797 | promoter | G/A | rs2839364 | 12.1% |
| 5 | −761 | promoter | C/T | unknown | |
| 6 | −738 | promoter | A/. | unknown | 12.3% |
| 7 | −100 | promoter | T/C | unknown | 50.0%^{b} |
| 8 | +2621 | intron | G/T | rs2186358 | 18.6% |
| 9 | +2766 | exon 2 | G/C | rs1051169 | 28.6%^{c} |
| 10 | +2821 | intron | T/C | unknown | 3.4% |
| 11 | +2852 | intron | A/G | rs2839356 | 9.2% |
| 12 | +2963 | intron | G/A | rs2839355 | 20.3%^{c} |
| 13 | +3029 | intron | T/A | unknown | 37.4% |
| 14 | +3055 | intron | G/A | unknown | <1% |
| 15 | +3492 | intron | G/A | rs6518303 | 13.2% |
| 16 | +3562 | intron | G/A | rs2300405 | 33.6% |
| 17 | +3916 | intron | C/T | rs2300404 | 35.8% |
| 18 | +3942 | intron | A/G | rs2300403 | 27.4%^{c} |
| 19 | +4416 | intron | G/A | unknown | |
| 20 | +4631 | intron | C/T | unknown | <1% |
| 21 | +5059 | intron | A/G | rs2239575 | 9.6% |
| 22 | +5128 | intron | C/T | rs881827 | 32.1% |
| 23 | +5185 | intron | G/A | rs2239574 | 34.9% |
| 24 | +5757 | 3′-UTR | C/T | rs9722 | 13.1% |
| 25 | +5759 | 3′-UTR | G/C | unknown | <1% |

^{a}The base changes representative of each polymorphism are indicated using the coding strand as a reference.
^{b}Not in Hardy-Weinberg equilibrium (genotyping by enzymatic digestion and sequencing).
^{c}Associated with cognitive decline in an ELDNOR population subgroup exhibiting an MMSE score greater than or equal to 25.

To date, of the 25 polymorphisms listed, 23 have been studied in an ELDNOR population subgroup (see materials and methods) encompassing 254 individuals exhibiting an MMSE (Mini Mental State Examination) score greater than or equal to 25. This initial screening made it possible to demonstrate three polymorphisms (rs1051169, rs2839355 and rs2300403) located within, or in proximity of, intron 3, which are significantly associated with a more marked cognitive decline.

The study of these three polymorphisms was extended to the entire ELDNOR population (table 4). Only two polymorphisms, rs2839355 and rs2300403, showed a significant effect of similar amplitude. Thus, for the rs2000403 polymorphism, it was shown that individuals carrying the GG genotype had an MMSE score 1.7 points lower than individuals carrying at least one A allele (table 4).

TABLE 4
Impact of polymorphisms rs1051169, rs2839355 and rs2000403
on cognitive decline in the ELDNOR population

| rs1051169 | | | rs2839355 | | | rs2000403 | | |
|---|---|---|---|---|---|---|---|---|
| | n | MMSE | | n | MMSE | | n | MMSE |
| CC + CG | 712 | 21.3 ± 5.1 | GG + AG | 742 | 21.3 ± 5.1 | AA + AG | 700 | 21.4 ± 5.1 |
| GG | 71 | 20.2 ± 5.0 | AA | 41 | 19.7 ± 5.0 | GG | 83 | 19.7 ± 4.9 |
| ΔMMSE | | −1.1 | ΔMMSE | | −1.6 | ΔMMSE | | −1.7 |
| p | | NS | p | | 0.05 | p | | 0.004 |

However, since these two polymorphisms are in complete linkage-disequilibrium, and for reasons of statistical power, the subsequent studies were restricted to the rs2300403 polymorphism.

Thus, in the Limeil-Brévannes population (see materials and methods), the GG genotype of the rs2300403 polymorphism is associated with a four-fold increase in the risk of exhibiting cognitive decline (p=0.003; table 5(b)).

TABLE 5
Genotypic distribution of the rs2000403 polymorphism in populations
of patients suffering from dementias or exhibiting cognitive decline
and controls: (a) ELDNOR population; (b) Limeil-Brévannes
population (LV); (c) combination of the two populations.
(a) ELDNOR

| | | | | | OR^{d} | |
|---|---|---|---|---|---|---|
| | | Genotype distribution^{b} | | | GG versus | |
| | n | AA | AG | GG | AG + AA | p^{d} |
| Demented | 187 | 98 | 71 | 18 | 2.5 | 0.08 |
| cases | | (0.52) | (0.38) | (0.10) | [0.9-7.2] | |
| Controls^{b} | 145 | 77 | 63 | 5 | | |
| | | (0.53) | (0.43) | (0.04) | | |

(b) LV

| | | | | | OR^{d} | |
|---|---|---|---|---|---|---|
| | | Genotype distribution^{a} | | | GG versus | |
| | n | AA | AG | GG | AG + AA | p^{d} |
| Demented | 125 | 62 | 42 | 21 | 2.4 | 0.08 |
| cases | | (0.50) | (0.34) | (0.17) | [0.9-6.5] | |
| Cognitive | 36 | 15 | 13 | 8 | 4.1 | 0.003 |
| decline | | (0.42) | (0.36) | (0.22) | [1.6-10.2] | |
| Controls | 115 | 56 | 51 | 8 | | |
| | | (0.49) | (0.44) | (0.07) | | |

(c) Both

| | | | | | OR^{d} | |
|---|---|---|---|---|---|---|
| | | Genotype distribution^{b} | | | GG versus | |
| | n | AA | AG | GG | AG + AA | p^{d} |
| Demented | 312 | 160 | 113 | 39 | 2.4 | 0.04 |
| cases | | (0.51) | (0.36) | (0.13) | [1.2-4.7] | |
| Controls^{b} | 260 | 133 | 114 | 13 | | |
| | | (0.51) | (0.44) | (0.05) | | |

^{a}p = 0.037;
^{b}p = 0.08;
^{c}p = 0.004;
^{d}Adjusted with respect to age and sex.

In conclusion, the S100β gene appears to be a genetic determinant of cognitive decline.

The impact of the rs2000403 polymorphism on the risk of developing dementia was also studied. It was observed that the GG genotype was associated with an increase in the risk of developing this type of pathology in both the ELDNOR and the Limeil-Brévannes populations. This increase did not reach the threshold of significance in these two populations taken separately due to the low percentage of the genotype considered and to the restricted size of the populations studied. Nevertheless, this increase in risk becomes significant when the two populations are combined (table 5(c) above).

In order to reinforce this result, the impact of the rs2000403 polymorphism on the risk of developing Alzheimer's disease was studied. In the total population (see materials and methods), no significant effect of the GG genotype was demonstrated. However, since the ELDNOR and Limeil-Brévannes populations of patients with dementias are significantly older, the population studied was stratified as a function of age. It is thus apparent that the GG genotype was again associated with a more than two-fold increase in the risk of developing Alzheimer's-type dementia in the most elderly (table 6).

Finally, in order to demonstrate a potential link between the production of the Cog⁴⁷ isoform and the rs2000403 polymorphism, the cerebral samples previously studied were genotyped for this polymorphism. In the control population, no association was demonstrated between the rs2000403 polymorphism and the quantity of Cog⁴⁷ mRNA. In the afflicted population, however, an 77% increase in the quantity of Cog⁴⁷ mRNA was observed for individuals carrying the GG genotype compared to those carrying at least one A allele (table 7).

TABLE 6
Genotypic distribution of the rs2000403 polymorphism in populations
of patients suffering from Alzheimer's disease and controls:
(a) for the entire population; (b) for the oldest population
(defined by the median age of the population).
(a) rs2000403

| | | | | | OR^{c} | |
|---|---|---|---|---|---|---|
| | | Genotype distribution^{a} | | | GG versus | |
| | n | AA | AG | GG | AG + AA | p^{c} |
| AD cases | 608 | 285 | 261 | 62 | 1.34 | 0.16 |
| | | (0.47) | (0.43) | (0.10) | [0.88-2.04] | |
| Controls | 499 | 242 | 218 | 39 | | |
| | | (0.48) | (0.44) | (0.08) | | |

(b) rs2000403

| | | | | | OR^{c} | |
|---|---|---|---|---|---|---|
| | | Genotype distribution^{a} | | | GG versus | |
| In the oldest | n | AA | AG | GG | AG +AA | p^{c} |
| AD cases | 284 | 129 | 122 | 33 | 2.21 | 0.035 |
| | | (0.45) | (0.43) | (0.12) | [1.06-4.62] | |
| Controls | 173 | 81 | 82 | 10 | | |
| | | (0.47) | (0.47) | (0.06) | | |

^{a}p = 0.38;
^{b}p = 0.10;
^{c}Adjusted with respect to age and sex.

TABLE 7
Expression levels of various mRNA arising from the S100β gene
in the control population and in the population suffering from Alzheimer's
disease as a function of the rs2000403 genotype.
(a) Controls

| | AA + AG | GG | | |
|---|---|---|---|---|
| n | 79 | 6 | | p |
| Normal S100β | 1.22 | 1.04 | −15.0% | NS |
| mRNA | [0.63-2.36] | [0.59-1.85] | | |
| Cog⁴⁷ mRNA | 0.014 | 0.015 | +4.6% | NS |
| | [0.008-0.026] | [0.010-0.022] | | |
| Cog⁴⁷/S100β | 0.012 | 0.014 | +23.0% | NS |
| ratio | [0.003-0.041] | [0.006-0.037] | | |

(b) Afflicted

| | AA + AG | GG | | |
|---|---|---|---|---|
| n | 68 | 13 | | p |
| Normal S100β | 0.81 | 0.57 | −30.6% | 0.06 |
| mRNA | [0.44-1.51] | [0.23-1.37] | | |
| Cog⁴⁷ mRNA | 0.019 | 0.033 | +77.4% | 0.02 |
| | [0.010-0.033] | [0.017-0.064] | | |
| Cog⁴⁷/S100β | 0.023 | 0.058 | +155.5% | 0.05 |
| ratio | [0.007-0.076] | [0.011-0.309] | | |

In summary, it was observed that:
the mRNA arising from the S100β gene undergoes novel alternative splicing, leading to the formation of the Cog⁴⁷ isoform;
the corresponding protein potentially differs from the normal S100β form by the last 48 amino acids;
the quantity of Cog⁴⁷ mRNA is more than 40% higher in the cerebral tissue of afflicted patients compared to the controls, whereas the quantity of Sl003 mRNA is almost 37% lower;
a high level of Cog⁴⁷ mRNA in cerebral tissue is associated with a more than three-fold increase in the risk of developing Alzheimer's disease;
the level of Cog⁴⁷ expression is correlated with the extent of neurofibrillary degeneration in the cerebral tissue of afflicted patients as well as to microglial cell activation.

In parallel, it was demonstrated at the genetic level that:
the rs2000403 polymorphism, localized in intron 3 of the S100β gene, is a genetic determinant of cognitive decline, with the GG genotype itself being associated with both a reduction in cognitive performance measured by the MMSE and an increase in the risk of developing cognitive decline;
the rs2000403 polymorphism is a genetic determinant of the risk of developing dementia, in particular Alzheimer's disease, with the GG genotype being associated with a two-fold increase in this risk;
the rs2000403 polymorphism is associated with an increase in the quantity of Cog⁴⁷ mRNA in the cerebral tissue of afflicted patients, with the GG genotype increasing this quantity by 77% compared to individuals not carrying this genotype.

In light of the preceding results, it appears that the Cog⁴⁷ isoform is a determinant of cognitive decline and dementia, in particular Alzheimer's disease. By increasing the quantity of Cog⁴⁷, the rs2000403 polymorphism thus accentuates the risk of exhibiting cognitive decline and then dementia.

1-29. (canceled)

30. A protein named Cog⁴⁷ represented by the sequence SEQ ID NO 1 having a sequence of amino acids that is different than that of the normal S100β protein.

31. A monoclonal or polyclonal antibody, or a fragment of said antibody, binding specifically to the protein according to claim 30 by affinity with the sequence of amino acids that is different than that of the normal S100β protein.

32. A diagnostic kit for determining the presence of the Cog⁴⁷ protein represented by the sequence SEQ ID NO 1 in a human sample, comprising an antibody, or fragment thereof, according to claim 31.

33. A method for determining the presence of a Cog⁴⁷ protein represented by the sequence SEQ ID NO 1 in a human sample comprising the following steps:
allowing the sample to be analyzed to react with a first antibody according to claim 31,
allowing the sample to be analyzed to react with a second antibody specifically directed against the antibody according to claim 31,
washing, and
detecting the quantity of Cog⁴⁷ protein in the sample.

34. The method according to claim 33, wherein the antibody is a monoclonal antibody.

35. An in vitro method for identifying a molecule capable of interacting with the Cog⁴⁷ protein comprising the following steps:
a) bringing together a suitable cellular model with the Cog⁴⁷ protein according to claim 30 and said molecule to be identified in order to characterize the quantity of phosphorylated tau proteins using at least one marker specific for tau protein phosphorylation, and/or
b) bringing together a suitable cellular model with the Cog⁴⁷ protein according to claim 30 and said molecule to be identified in order to characterize the level of expression of at least one marker for microglial inflammation,
c) when:
the quantity of phosphorylated tau proteins characterized in step a) is less than the quantity of phosphorylated tau proteins characterized in the absence of said molecule, and/or
the level of expression of said at least one marker for microglial inflammation characterized in step b) is less than the level of expression of said marker characterized in the absence of said molecule,
identifying said molecule capable of interacting with the Cog⁴⁷ protein.

36. The identification method according to claim 35, wherein said at least one marker specific for tau protein phosphorylation is an antibody directed against a phosphorylated epitope of the tau protein, this epitope being characteristic of neuronal degeneration.

37. The identification method according to claim 35, wherein said at least one marker for microglial inflammation is a cytokine.

38. An isolated and purified mRNA polynucleotide encoding the Cog⁴⁷ protein according to claim 30, represented by the sequence SEQ ID NO 2.

39. A diagnostic kit for determining the presence of the Cog⁴⁷ protein represented by the sequence SEQ ID NO 1 in a human sample, comprising an oligonucleotide comprising 20 to 25 bp complementary to the mRNA according to claim 38.

40. A method for diagnosing a predisposition to cognitive decline, to conversion to dementia or to already existing dementia comprising the determination of the presence of the polynucleotide according to claim 38.

41. An interfering mRNA polynucleotide that specifically targets the mRNA according to claim 38.

42. A method for inhibiting or decreasing expression of the Cog⁴⁷ protein in a subject, comprising administering to said subject the interfering mRNA polynucleotide according to claim 41.

43. A DNA polynucleotide capable of expressing the interfering mRNA polynucleotide according to claim 41 in a cell.

44. A method for inhibiting or decreasing expression of the Cog⁴⁷ protein in a subject, comprising administering to said subject the DNA polynucleotide according to claim 43.

45. A Cog⁴⁷ protein expression vector comprising at least the DNA sequence complementary to the sequence SEQ ID NO 2 of the mRNA polynucleotide encoding the Cog⁴⁷ protein according to claim 38 and elements allowing the expression of said protein in a prokaryotic host cell with the purpose of producing a recombinant protein.

46. A transgenic mouse comprising in its genome the nucleotide sequence required to express the Cog⁴⁷ protein according to claim 30 represented by the sequence SEQ ID NO 1 with the purpose of developing a model for studying the involvement of Cog⁴⁷ in pathological processes and potential therapeutic targets.

47. A method for diagnosing a predisposition to cognitive decline, to conversion to dementia or to already existing dementia comprising the determination of the presence of a polymorphism located at a position with respect to the transcription initiation site of said gene selected in the group consisting of the guanidine nucleotide that is +2766, the adenine nucleotide that is +2963, and the guanidine nucleotide that is +3942.

48. The method according to claim 47, wherein the polymorphism is located at the guanidine nucleotide that is +2766 with respect to the transcription initiation site of the human S100 gene and the guanidine nucleotide is replaced by a cytosine nucleotide.

49. The method according to claim 47, wherein the polymorphism is located at the adenine nucleotide that is +2963 with respect to the transcription initiation site of the human S100β gene, and the guanidine nucleotide is replaced by an adenine nucleotide.

50. The method according to claim 47, wherein the polymorphism is located at the guanidine nucleotide that is +3942 with respect to the transcription initiation site of the human S100β gene, and the guanidine nucleotide is replaced by an adenine nucleotide.

51. A diagnostic kit for determining the presence in a human sample of the human S100β gene according to claim 47, comprising at least one oligonucleotide of 20 to 25 bp complementary to a polymorphism selected in the group consisting of the polymorphism located at the thymidine nucleotide that is −100 with respect to the transcription initiation site of the human S100β gene in which the thymidine nucleotide is replaced by a cytosine nucleotide, the polymorphism located at the guanidine nucleotide that is +2766 with respect to the transcription initiation site of the human S100β gene in which the guanidine nucleotide is replaced by a cytosine nucleotide, the polymorphism located at the adenine nucleotide that is +2963 with respect to the transcription initiation site of the human S100β gene in which the guanidine nucleotide is replaced by an adenine nucleotide, and the polymorphism located at the guanidine nucleotide that is +3942 with respect to the transcription initiation site of the human S100β gene in which the guanidine nucleotide is replaced by an adenine nucleotide.


SEQUENCE LISTING (3 sequences)

SEQ ID NO: 1 - protein, 94 aa, Homo sapiens
  Proteine Cog47
MSELEKAMVALIDVFHQYSGREGDKHKLKKSELKELINNELSHFLERWSLPMLPRLVSNFLCSSYPPALASQSAGITGNQRAGGCGQSHGNTGQ

SEQ ID NO: 2 - RNA, 1189 nt, Homo sapiens
  COG47 mRNA
ugccgcccaggacccgcagcagagacgacgccugcagcaaggagaccaggaaggggugagacaaggaagaggaugucugagcuggagaaggccaugguggcccucaucgacguuuuccaccaauauucuggaagggagggagacaagcacaagcugaagaaauccgaacucaaggagcucaucaacaaugagcuuucccauuucuuagagagauggagucucccuauguugcccaggcuggucucaaacuuccugugcucaaguuauccuccugccuuagcuucccaaagugcugggauuacaggaaaucaaagagcaggagguuguggacaaagucauggaaacacuggacaaugauggagacggcgaaugugacuuccaggaauucauggccuuuguugccaugguuacuacugccugccacgaguucuuugaacaugagugagauuagaaagcagccaaaccuuuccuguaacagagacggucaugcaagaaagcagacagcaagggcuugcagccuaguaggagcugagcuuuccagccguguuguagcuaauuaggaagcuugauuugcuuugugauugaaaaauugaaaaccucuuuccaaaggcuguuuuaacggccugcaucauucuuucugcuauauuaggccuguguguaagcugacuggccccagggacucuuguuaacaguaacuuaggagucaggucucagugauaaagcgugcaccgugcagcccgccauggccguguagacccuaacccggagggaacccugacuacagaaauuaccccggggcacccuuaaaacuuccacuaccuuuaaaaaacaaagccuuauccagcauuauuugaaaacacugcuguucuuuaaaugcguuccucauccaugcagauaacagcugguuggccgguguggcccugcaagggcgugguggcuucggccugcuucccgggaugcgccugaucaccaggugaacgcucagcgcuggcagcguccuggaaaaagcaacuccaucagaacucgcaauccgagccagcucugggggcuccagcguggccuccgugacccaugcgauucaagucgcggcugcaggauccuugccuccaacgugccuccagcacaugcggcuuccgagggcacuaccgggggcucugagccaccgcgagggccugcguucaauaaaaag

SEQ ID NO: 3 - RNA, 1095 nt, Homo sapiens
  ARNm de S100beta normal
ugccgcccaggacccgcagcagagacgacgccugcagcaaggagaccaggaaggggugagacaaggaagaggaugucugagcuggagaaggccaugguggcccucaucgacguuuuccaccaauauucuggaagggagggagacaagcacaagcugaagaaauccgaacucaaggagcucaucaacaaugagcuuucccauuucuuagaggaaaucaaagagcaggagguuguggacaaagucauggaaacacuggacaaugauggagacggcgaaugugacuuccaggaauucauggccuuuguugccaugguuacuacugccugccacgaguucuuugaacaugagugagauuagaaagcagccaaaccuuuccuguaacagagacggucaugcaagaaagcagacagcaagggcuugcagccuaguaggagcugagcuuuccagccguguuguagcuaauuaggaagcuugauuugcuuugugauugaaaaauugaaaaccucuuuccaaaggcuguuuuaacggccugcaucauucuuucugcuauauuaggccuguguguaagcugacuggccccagggacucuuguuaacaguaacuuaggagucaggucucagugauaaagcgugcaccgugcagcccgccauggccguguagacccuaacccggagggaacccugacuacagaaauuaccccggggcacccuuaaaacuuccacuaccuuuaaaaaacaaagccuuauccagcauuauuugaaaacacugcuguucuuuaaaugcguuccucauccaugcagauaacagcugguuggccgguguggcccugcaagggcgugguggcuucggccugcuucccgggaugcgccugaucaccaggugaacgcucagcgcuggcagcguccuggaaaaagcaacuccaucagaacucgcaauccgagccagcucugggggcuccagcguggccuccgugacccaugcgauucaagucgcggcugcaggauccuugccuccaacgugccuccagcacaugcggcuuccgagggcacuaccgggggcucugagccaccgcgagggccugcguucaauaaaaag